(19) United States
(12) Patent Application Publication
(10) Pub. No.: US 2011/0208034 A1
(43) Pub. Date: Aug. 25, 2011

(54) MEDICAL EXAMINATION AND/OR TREATMENT APPARATUS

(76) Inventors: Oliver Heid, Erlangen (DE); Timothy Hughes, Erlangen (DE)

(21) Appl. No.: 13/026,413

(22) Filed: Feb. 14, 2011

(30) Foreign Application Priority Data
Feb. 22, 2010 (DE) 10 2010 008 778.5

(51) Int. Cl. A61B 5/05

(52) U.S. Cl. 600/407

(57) ABSTRACT

A medical examination and/or treatment apparatus having an apparatus for position acquisition assigned to a control facility has at least one transmitter that is disposed on a movable part that can be controlled by way of the control facility and transmits a signal and at least one receiver disposed on the movable part for receiving a signal reflected by an external object, the apparatus for position acquisition being configured to determine the distance of the external object from the at least one movable part from the transmitted and received signal, wherein the signal is an FMCW signal of a predefined frequency or frequency range.

### CROSS-REFERENCE TO RELATED APPLICATIONS

This application claims priority to DE Patent Application No. 10 2010 008 778.5 filed Feb. 22, 2010. The contents of which is incorporated herein by reference in its entirety.

### TECHNICAL FIELD

The invention relates to a medical examination and/or treatment apparatus having an apparatus for position acquisition assigned to a control facility comprising at least one transmitter that is disposed on a movable part that can be controlled by way of the control facility and transmits a signal and at least one receiver disposed on the movable part for receiving a signal reflected by an external object, the apparatus for position acquisition being configured to determine the distance of the external object from the at least one movable part from the transmitted and received signal.

### BACKGROUND

It is known that robots that move largely automatically can be used in medical examinations and/or treatments. The robots generally used here are industrial robots with a number of, in particular six, degrees of freedom, which support a person being treated in a wide range of situations, for example during the recording of an image of an examination and/or treatment region, such as e.g. when performing an angiography, or during invasive interventions. The static and dynamic objects present with the robot in a room, such as a patient table for example or a person being treated or a patient, mean that there is a risk of the robot colliding with an object, which can cause damage or injury to the object.

### SUMMARY

According to various embodiment, a medical examination and/or treatment apparatus can be specified, with which the risk of a movable part colliding with an external object is reduced.

According to an embodiment, a medical examination and/or treatment apparatus may have an apparatus for position acquisition assigned to a control facility comprising at least one transmitter that is disposed on a movable part that can be controlled by way of the control facility and transmits a signal and at least one receiver disposed on the movable part for receiving a signal reflected by an external object, the apparatus for position acquisition being configured to determine the distance of the external object from the at least one movable part from the transmitted and received signal, characterized in that the signal is an FMCW signal of a predefined frequency or frequency range.

According to a further embodiment, the movement of the movable part can be controlled by the control facility as a function of the distance determined. According to a further embodiment, the external object may be movable and the movement of the external object can be controlled by the control facility as a function of the distance determined. According to a further embodiment, a number of transmitters may be provided, with at least one receiver being assigned to each transmitter, and the frequencies or frequency ranges of the signals sent by the transmitter being different from one another and the respectively assigned receiver only receiving signals in the specified frequency or frequency range of the transmitter assigned to it. According to a further embodiment, the transmitter and/or the receiver can be configured as an array.

### BRIEF DESCRIPTION OF THE DRAWINGS

Further advantages, features and details will emerge from the exemplary embodiment described in the following and with reference to the drawings.

FIG. 1 shows an embodiment of a medical examination and/or treatment apparatus in a basic diagram.

### DETAILED DESCRIPTION

According to various embodiments, the problem is resolved by a medical examination and/or treatment apparatus of the type mentioned in the introduction, which is distinguished in that the signal is an FMCW signal of a predefined frequency or frequency range.

The medical examination and/or treatment apparatus according to various embodiments, which may be for example an industrial robot with six axes that can be controlled independently of one another by way of a control facility, in other words six movable parts, measures a distance between at least one of its movable parts and an external object by way of the frequency shift of an FMCW signal. The signals transmitted by a transmitter according to various embodiments are then frequency-modulated signals, in other words the signal changes its frequency continuously in a defined frequency range, e.g. in a sinusoidal or saw-tooth manner. In principle an FMCW signal has a base frequency, which is overlaid with a second frequency that changes over time.

The frequency shift of the FMCW signal, which results from the frequency difference between an FMCW signal transmitted by the transmitter and one received by the receiver after reflection off an external object, therefore allows conclusions to be drawn about the distance of the corresponding part from an external object. The difference between the transmitted and received frequency of the FMCW signal is proportional to the distance between transmitters or receivers disposed on the movable part and the external object. The distance between the movable part and the external object can thus be determined easily from the frequency shift. An external object here can be understood to be both a static (immovable) and also a dynamic (movable) object.

The FMCW signal transmitted by the transmitter has a previously set frequency or a previously set frequency range. The FMCW signal can be sensitive to clothing or the body for example as a result of the selection and setting of the frequency or frequency range. As far as non-human objects in the room are concerned, the FMCW signal transmitted by the transmitter is of course selected so that it discloses such objects or is reflected by them. The FMCW signal can be set to any frequencies or frequency ranges.

A transmitter can advantageously be assigned a receiver, the assignment being made according to the frequencies or frequency ranges transmitted by the transmitter and the frequencies or frequency ranges that can be received by the receiver. A transmitter therefore transmits FMCW signals of a certain frequency or frequency range and the assigned receiver is set so that it can only receive FMCW signals with these frequencies or frequency ranges of the transmitter assigned to it.

The positioning of the transmitter and receiver on at least one movable part can be selected in an arbitrary manner here. It can be particularly advantageous to position them on movable parts, which are subject to a particular risk of collision, in other words movable parts, which are by nature disposed particularly close to the external object or can be moved to such a position. Thus for example in the case of a six-axis robot it can be particularly advantageous to position the transmitter and receiver on parts which have a particularly large number of degrees of freedom and thus can be moved over large distances in the room. Of course a number of transmitters and a corresponding number of receivers can also be disposed on a movable part. In some instances the transmitter can also be provided in a place other than directly on a movable part and can communicate with one or a number of receivers disposed on a movable part, thereby resulting likewise in position acquisition and therefore measurement of the distance of the movable part in relation to an external object. It is important that the frequency shift of the FMCW signal transmitted by a transmitter, which is registered by a receiver and furthermore the apparatus for position acquisition, allows a distance or change in a distance of the movable part from an external object and thus the possibility of a collision to be determined.

The movement of a movable part can expediently be controlled by the control facility as a function of the distance determined. The control facility thus decides, as the movable part approaches an external object, which can be determined by way of the frequency shift of the FMCW signal described above by way of the distance determination apparatus, whether the movable part is at a sufficiently great distance from the external object, so that there is no risk of collision, or whether in some instances the movement of the movable part must be restricted or stopped completely to avoid a collision. It is also conceivable in some instances for a movable part, when it actively approaches the external object, which can in particular also be a person, to be moved away from this so that the risk of collision is equally avoided.

It can be advantageous if the external object is movable and the movement of the external object can be controlled by the control facility as a function of the distance determined. This is so when the external object is an apparatus that can likewise be moved in the room through at least one degree of freedom. If a movable part approaches in a manner that increases the possibility of a collision, it is thus also possible to move the external object in a controlled manner by way of the control facility according to its degrees of freedom, so that collision of the movable object with this is prevented. This can take place in addition to the above option, where the movement of the movable part is controlled by the control facility in the event of a possible collision so that a collision is avoided. A movable external object can be for example a height-adjustable patient table, which communicates by way of suitable connecting means with the control facility and can be moved in a controlled manner by said control facility.

According to an embodiment, it is possible for a number of transmitters to be provided, with at least one receiver being assigned to each transmitter and the frequencies or frequency ranges of the signals sent by the transmitter being different from one another and the respectively assigned receiver only receiving signals in the specified frequency or frequency range of the transmitter assigned to it. It is thus possible in the case of a number of transmitters and receivers disposed on one or distributed over different movable parts to assign a specified receiver to a specified transmitter, the transmitter communicating exclusively with said receiver. It is correspondingly possible to change the distance by moving a specified movable part in relation to an external object by way of the paired assignment of transmitter and receiver over the specified frequency range. This also excludes interference with one or more optionally further medical examination and/or treatment apparatuses, since the transmitters and receivers utilized by these expediently communicate in a different frequency range.

Transmitters and/or receivers can advantageously be configured as an array. This allows signal amplification of the transmitted FMCW signal by overlaying (constructive interference). The sensitivity of a receiver to a signal is also increased. This allows better transmit and receive properties of the transmitter and receiver respectively to be achieved.

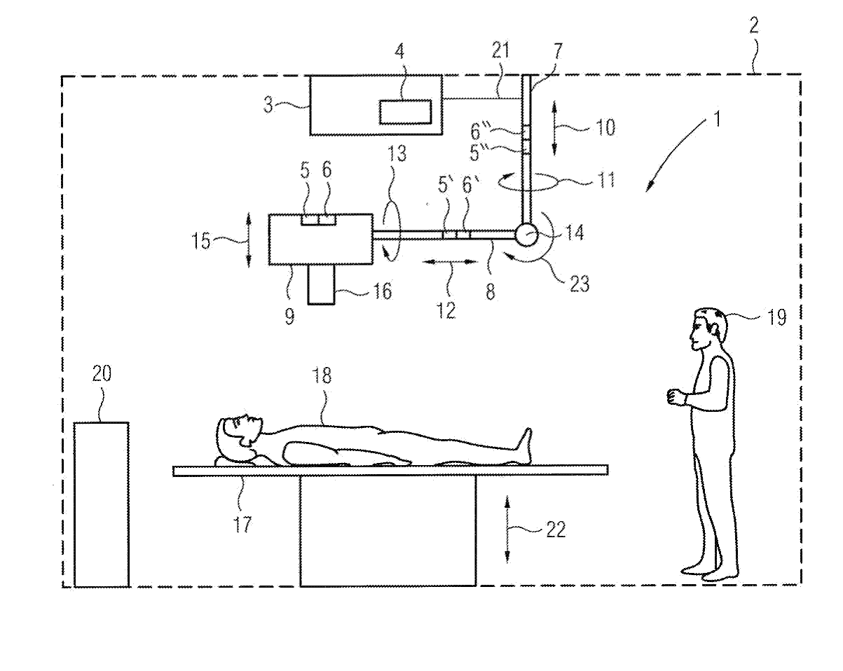

The figure shows a basic diagram of a medical examination and/or treatment apparatus 1 according to various embodiments in an examination and/or treatment room 2 marked with a broken line. Associated with the medical examination and/or treatment apparatus 1 is a control facility 3 and an apparatus for position acquisition 4 assigned thereto. Assigned to the apparatus for position acquisition 4 are three transmitters 5, 5′, 5″ transmitting FMCW signals and correspondingly three receivers 6, 6′, 6″ receiving the FMCW signals reflected by an external object. The transmitters 5, 5′, 5″ and receivers 6, 6′, 6″ are disposed on movable parts 7, 8, 9. The movable part 7 here has two degrees of freedom, as it can be moved along the arrow 10 in its vertical position and along the arrow 11 in a rotatable manner about its axis. The same applies to the movable part 8, as it can be displaced along the arrow 12 lengthwise, in other words horizontally, and can be rotated along the arrow 13 about its axis. The connection between the movable part 7 and the movable part 8 is achieved by way of the articulation 14, which also allows pivotablity along the arrow 23 of the movable part 8 and the movable part 9 disposed thereon, which is disposed on the movable part 8 in such a manner that it can be displaced vertically along the arrow 15. The movable part 9 also comprises a beam source 16, by way of which radiation is emitted for example for examination purposes onto a patient 18 lying on a patient couch 17. The beam source 16 can be embodied for example as an x-ray tube. A corresponding x-ray detector is not shown in FIG. 1, although it is of course present. The beam source 16 should not be confused with the transmitters 5, 5′, 5″. Also present in the treatment room 2 are a person 19 being treated and a static object in the form of a fixed cabinet 20.

If the transmitter 5 now emits an FMCW signal of a predefined frequency or frequency range for example, said frequency signal is reflected by the patient 18 for example, resulting in a frequency shift of the FMCW signal, which is detected by the receiver 6, which receives the reflected FMCW signal, in conjunction with the apparatus for position acquisition 4 or its control or processing facility 3. The proportionality described above between the frequency shift and distance allows the distance between the movable part 9 and the patient to be determined from the frequency shift of the FMCW signal. The same applies to the movable parts 7, 8, which likewise have transmitters 5″, 5′ and receivers 6″, 6′ disposed in pairs. It is therefore generally possible, for each of the movable parts 7, 8, 9, to determine the distance from an external object present in the treatment room 2, here including the patient table 17, the patient 18, the person 19 being treated and the cabinet 20.

The respective transmitters 5, 5′, 5″ are assigned to the respective receivers 6, 6′, 6″ by way of different FMCW signals transmitted by the transmitters 5, 5′, 5″, which differ in frequency and frequency range. The receiver 6, 6′, 6″ assigned to the respective transmitter 5, 5′, 5″ here is configured in such a manner that it can only receive FMCW signals of the corresponding transmitter 5, 5′, 5″ assigned to it. This means that the exemplary transmitter/receiver pairs 5-6, 5′-6′, 5″-6″ communicate with one another at respectively different frequencies or frequency ranges. Transmitters and receivers are each configured as arrays.

A movement of one of the movable parts 7, 8, 9 in the direction of the patient 18 for example, which increases the risk of collision with the latter or would result in such, can be detected by the measurement performed by the apparatus for position acquisition 4 of the distance between the respective movable part 7, 8, 9 and the external object, in this instance the patient 18. In the event of a collision the control facility 3 intervenes in the movement of the movable part 7, 8, 9 such that its movement is restricted or stopped completely. The control facility 3 is therefore connected to the movable parts 7, 8, 9 by way of a communication means, in this instance in the form of a cable 21, to control said movable parts 7, 8, 9.

It may be particularly advantageous for an external object, such as the patient table 17 for example, also to be movable (see the capacity for vertical movement of the patient table 17 along the arrow 22), so that the control facility 3 is likewise configured to control the patient table 17 and can control its movement. In the event of a possible collision of one of the movable parts 7, 8, 9 with the patient table 17, it is therefore additionally possible here to displace the patient table 17 as well as the movable parts 7, 8, 9 to avoid a collision.

Similarly in certain instances, in particular before and after an examination or treatment, it is possible for an approach of the person 19 being treated for example toward one of the movable parts 7, 8, 9, which would likewise result in a collision, to be identified by means of the distance measurement performed by the apparatus for position acquisition 4 and for the control facility 3 also to initiate a corresponding movement of the movable part(s) 7, 8, 9 away from the person 19 being treated. This can be advantageous, for example, also to facilitate the descent of a patient 18 from the patient table 17 after completion of an examination. Of course during all movements of the movable parts 7, 8, 9 to avoid collision and brought about by the control facility 3, all the other external objects present in the room (patient table 17 and patient 19 or person 19 being treated and cabinet 20) are taken into account, in other words the movement is executed so that no collision with said objects takes place in the process.

### LIST OF REFERENCE CHARACTERS

1 Examination and/or treatment apparatus
2 Examination and/or treatment room
3 Control facility
4 Apparatus for position acquisition
5 Transmitter
5′ Transmitter
5″ Transmitter
6 Receiver
6′ Receiver
6″ Receiver
7 Part
8 Part
9 Part
10 Arrow
11 Arrow
12 Arrow
13 Arrow
14 Articulation
15 Arrow
16 Beam source
17 Patient couch
18 Patient
19 Person
20 Cabinet

What is claimed is:

1. A medical examination and/or treatment apparatus comprising:
an apparatus for position acquisition assigned to a control facility comprising at least one transmitter that is disposed on a movable part that can be controlled by way of the control facility and transmits a signal, and
at least one receiver disposed on the movable part for receiving a signal reflected by an external object, the apparatus for position acquisition being configured to determine the distance of the external object from the at least one movable part from the transmitted and received signal, wherein the signal is an FMCW signal of a predefined frequency or frequency range.

2. The apparatus according to claim 1, wherein the movement of the movable part can be controlled by the control facility as a function of the distance determined.

3. The apparatus according to claim 1, wherein the external object is movable and the movement of the external object can be controlled by the control facility as a function of the distance determined.

4. The apparatus according to claim 1, wherein a plurality of transmitters are provided, with at least one receiver being assigned to each transmitter, and the frequencies or frequency ranges of the signals sent by the plurality of transmitters being different from one another and the respectively assigned receiver only receiving signals in the specified frequency or frequency range of a transmitter assigned to it.

5. The apparatus according to claim 1, wherein at least one of the transmitter and the receiver are configured as an array.

6. The apparatus according to claim 1, wherein the movable part has two degrees of freedom.

7. The apparatus according to claim 1, further comprising at least two movable parts and a connection between the movable parts comprising an articulation, which also allows pivotablity of a movable part.

8. The apparatus according to claim 1, wherein the movable part comprises a beam source, by way of which radiation is emitted.

9. The apparatus according to claim 1, wherein a reflection of the frequency signal results in a frequency shift of the FMCW signal, which is detected by the receiver.

10. The apparatus according to claim 1, wherein the frequency shift allows for determining the distance between the movable part and a patient.

11. The apparatus according to claim 1, wherein in the event of a possible collision of the apparatus and the external object, the control facility is configured to intervene in the movement of the movable part such that its movement is restricted or stopped completely.

12. The apparatus according to claim 3, wherein in the event of a possible collision of the apparatus and the external object, the control facility is configured to displace the external object.

13. The apparatus according to claim 12, further comprising
a moveable patient table for supporting the external object.

14. A method of operating a medical examination and/or treatment apparatus at least one transmitter that is disposed on a movable part of the apparatus that can be controlled, the method comprising:
transmitting by the at least one transmitter which is disposed on the movable part a signal,
receiving by at least one receiver disposed on the movable part the signal reflected by an external object,
determining the distance of the external object from the at least one movable part from the transmitted and received signal, wherein the signal is an FMCW signal of a predefined frequency or frequency range,
controlling said movable part depending on the determined distance.

15. The method according to claim 14, wherein the external object is movable and the method comprises controlling a movement of the external object a function of the distance determined.

16. The method according to claim 14, wherein a plurality of transmitters are provided, with at least one receiver being assigned to each transmitter, and the frequencies or frequency ranges of the signals sent by the plurality of transmitters being different from one another and the respectively assigned receiver only receiving signals in the specified frequency or frequency range of a transmitter assigned to it.

17. The method according to claim 14, wherein a reflection of the frequency signal results in a frequency shift of the FMCW signal, which is detected by the receiver.

18. The method according to claim 17, wherein the frequency shift allows for determining the distance between the movable part and a patient.

19. The method according to claim 14, wherein if a possible collision of the apparatus and the external object is determined, then intervening in the movement of the movable part by the apparatus such that its movement is restricted or stopped completely.

20. The method according to claim 15, wherein if a possible collision of the apparatus and the external object is determined, then displacing the external object by the apparatus.
